(19) United States
(12) Patent Application Publication
(10) Pub. No.: US 2015/0202005 A1
(43) Pub. Date: Jul. 23, 2015

(54) System and Method To Control Surgical Energy Devices

(71) Applicant: OmniGuide, Inc., Cambridge, MA (US)

(72) Inventors: Vladimir Fuflyigin, Medford, MA (US); Lori Pressman, Cambridge, MA (US)

(21) Appl. No.: 14/599,984

(22) Filed: Jan. 19, 2015

Related U.S. Application Data
(60) Provisional application No. 61/929,343, filed on Jan. 20, 2014.

(51) Int. Cl. A61B 18/20; A61B 19/00

(52) U.S. Cl. CPC A61B 18/20; A61B 19/5202; A61B 2018/00642

(57) ABSTRACT

A system and method for optimizing treatment of tissue of a patient at a surgical site by directing at least a first wavelength of illuminating radiation to a target location on the tissue of the patient to cause at least one type of fluorophore associated with the tissue to fluoresce. At least a first surgical energy is directed to the target location at one of a plurality of power settings, at least one of the power settings capable of quenching fluorescence of the fluorophore. At least one of a plurality of system control settings is adjusted to alter the amount of quenching of the fluorophore, the system control settings including the plurality of power settings.

### CROSS-REFERENCE TO RELATED APPLICATION

This application claims priority to U.S. Provisional Application No. 61/929,343 filed 20 Jan. 2014.

### FIELD OF THE INVENTION

The invention relates to controlling tissue surgery based on tissue interactions with surgical energy devices such as lasers.

### BACKGROUND OF THE INVENTION

There are a number of surgical devices utilized by surgeons that deliver one or more types of energy to cut, coagulate, ablate, remove or otherwise treat tissue of a patient. Surgical energy devices include ultrasonic devices, electrosurgical devices utilizing monopolar and/or bipolar RF (Radio-Frequency) current, microwave and/or thermal energy devices, and higher-frequency electromagnetic radiation delivery devices such as lasers. Examples of medical laser systems utilizing hollow waveguides are provided by Temelkuran et al. in U.S. Pat. Nos. 7,167,622 and 7,331,954, and by Goell et al. in U.S. Pat. No. 8,280,212, all assigned to OmniGuide, Inc. of Cambridge, Mass.

Various imaging techniques assist surgeons to access and treat targeted tissue. As an example, a fluorescence endoscopy video system is described by Cline et al. in U.S. Pat. No. 6,821,245. Compounds that generate fluorescence when excited are referred to as fluorophores; exogenous fluorophores include certain photoactive drugs and dyes delivered to selected tissue, while endogenous fluorophores are normally present in most tissues. Fluorescence guidance may be particularly useful in minimally invasive procedures, where the surgical site is seen through an endoscope or other visualization system inside a body cavity under artificial illumination, making landmarks or distinguishing features otherwise evident during open-field surgery less visible to the surgeon.

During fluorescence-guided surgery, one or more fluorophores are “pumped”, that is, are energized to an excited state, with specific electromagnetic wavelengths to identify tissues which are to be treated or removed with a surgical tool, such as a scalpel, laser or electro-cautery device. For example, U.S. Patent Publication No. 2004/0142485 by Flower et al. describes the use of indocyanine green “ICG” to identify and then treat choroidal neovascularizations. See also U.S. Patent Publication No. 2013/0078188 by Tsien et al. In some procedures, another fluorophore is used to identify tissues which are not to be removed and are to be protected from inadvertent damage by the treatment device, such as described by Tsien et al. in U.S. Patent Publication No. 2012/0148499.

A difficulty in targeting desired tissue may arise when utilizing a non-visible energy beam such as a CO₂ laser beam. Positioning of an ultrasonic beam utilizing one or more light sources is disclosed by McCarty in U.S. Pat. No. 5,107,709. It is known to align non-visible laser beams utilizing phosphor screens available from LUMITEK International, Inc. of Ijamsville, Md., especially for bench testing and research purposes. Visible aiming beams utilized with CO₂ lasers are described by Michael Black in U.S. Pat. No. 5,420,882, by Temelkuran et al. in U.S. Pat. No. 7,331,954, by Shapira et al. in WO2006/135701 and by Gannot et al. in U.S. Patent Publication No. 2006/0052661, for example. However, there remains a need to optimize tissue treatment under actual conditions which may vary from patient to patient, and may vary among different tissues and locations within each patient.

It is therefore desirable to have an improved system and method to effectively treat selected tissue within a patient utilizing surgical energy devices.

### SUMMARY OF THE INVENTION

An object of the present invention is to locate the thermal footprint of a surgical energy device and adjust delivered energy as needed to minimize damage to non-targeted tissue.

Another object of the present invention is to estimate the thermal effects of full treatment on selected tissue by determining the amount of quenching of fluorescence in the tissue utilizing an initial, lower level of treatment.

A still further object of the invention is to utilize quenching of fluorescence to guide the use of surgical energy to enable more tissue sparing by controlling the energy thermal footprint.

This invention results from the realization that quenching of fluorescence in tissue can generate feedback to confirm or adjust one or more of the location, size and thermal profile of the thermal footprint of an energy beam to optimize delivery of energy to tissue during surgery.

This invention features a system having a radiation source of at least a first wavelength of illuminating radiation directable to a target location on tissue of a patient at a surgical site to cause at least one type of fluorophore associated with the tissue to fluoresce. The system further includes an energy source of at least a first surgical energy directable to the target location and having a plurality of power settings, at least one of the power settings being capable of quenching fluorescence of the fluorophore. The system preferably includes a processor capable of adjusting at least one of a plurality of system control settings to alter the amount of quenching of the fluorophore, the system control settings including the plurality of power settings.

In some embodiments, the first surgical energy is a beam of electromagnetic radiation such as a laser beam generated by the energy source. In certain embodiments, the processor includes at least one detector to detect quenching of fluorescence at the surgical site and at least one image processor capable of generating an image of the surgical site to depict changes in fluorescence. In some embodiments, the processor includes a controller to adjust the system control settings, such as at least one of power level, pulse rate, pulse width, pulse shape and duty cycle for delivery of electromagnetic radiation and/or altering the power and/or wavelength of the illuminating radiation. In certain embodiments, the controller is capable of moving the surgical energy to a different location on tissue at the surgical site.

In a number of embodiments, the radiation source provides at least a second wavelength of illuminating radiation to enable a user to see the surgical site. In certain embodiments, the system further includes at least one fluid source having at least two fluid control settings for at least one of fluid flow rate and temperature, and the adjustable system control settings include at least some of the fluid control settings.

This invention also features a method for optimizing treatment of tissue of a patient at a surgical site by directing at least a first wavelength of illuminating radiation to a target location on the tissue of the patient to cause at least one type of fluorophore associated with the tissue to fluoresce. The method further includes directing at least a first surgical energy from an energy source to the target location at one of a plurality of power settings, at least one of the power settings capable of quenching fluorescence of the fluorophore. At least one of a plurality of system control settings is adjusted to alter the amount of quenching of the fluorophore, the system control settings including the plurality of power settings.

### BRIEF DESCRIPTION OF THE DRAWINGS

In what follows, preferred embodiments of the invention are explained in more detail with reference to the drawings, in which:

FIG. 1 is a schematic diagram of a system according to the present invention optimizing radiation at a surgical site of a patient;

FIG. 2 is a schematic diagram of an alternative system according to the present invention being operated by a user such as a surgeon;

FIGS. 3A-3C are schematic illustrations of cross-sections of energy level delivered to tissue under certain normal, profile-sharpening and insufficient-to-quench power settings, respectively;

FIGS. 4A-4C are schematic illustrations of quench spots for the conditions of FIGS. 3A-3C;

FIGS. 5A-5C are schematic illustrations of fluorescence intensity for the conditions of FIGS. 3A-3C, respectively;

FIGS. 6A-6C represent schematic thermal profiles at target locations for the conditions of FIGS. 3A-3C, respectively;

FIG. 7A is a chart of thermal spread showing width and depth of thermal damage to tissue by various surgical energy devices;

FIG. 7B is a chart of penetration and absorption of various wavelengths of laser energy in pigmented and unpigmented tissue;

FIG. 8A is an illustration of spot size versus distance to tissue for an energy beam such as a diverging laser beam;

FIGS. 8B and 8C are illustrations of low power and higher power, respectively, with a CO₂ laser waveguide spaced relatively far from tissue;

FIGS. 8D and 8E are illustrations of low power and higher power, respectively, with a CO₂ laser waveguide spaced relatively close to tissue;

FIG. 9 is a schematic side cross-sectional view of the distal end of a surgical energy device such as a laser waveguide capable of delivering cooling fluid to alter a thermal profile according to the present invention;

FIG. 9A is a cross-sectional view along lines 9A-9A in FIG. 9;

FIG. 9B is a depiction of the beam-tissue interface of cooling fluid and an energy beam delivered from the device of FIG. 9;

FIG. 9C is a depiction similar to that of FIG. 9B further illustrating intensity of a Gaussian energy beam in radial cross-section at the beam-tissue interface;

FIGS. 10 and 10A are views similar to those of FIGS. 9 and 9A through an alternative waveguide for use according to the present invention;

FIG. 11 is a flow chart of steps to position delivery of surgical energy to determine the location where it strikes tissue;

FIG. 12 is a flow chart showing optimization of thermal footprint according to the present invention;

FIG. 13 is a flow chart indicating control of ranges for at least one system control setting; and

FIG. 14 is a flow chart of steps to calibrate a system according to the present invention.

### DETAILED DESCRIPTION OF THE PRESENTLY PREFERRED EMBODIMENTS

This invention may be accomplished by a system and method for optimizing delivery of energy to tissue of a patient at a surgical site by directing at least a first wavelength of illuminating radiation to a target location on the tissue of the patient to cause at least one type of fluorophore associated with the tissue to fluoresce. At least a first surgical energy is directed to the target location at one of a plurality of power settings, at least one of the power settings being capable of quenching fluorescence of the fluorophore. At least one of a plurality of system control settings is adjusted to alter the amount of quenching of the fluorophore, the system control settings including the plurality of power settings.

In other words, systems and methods according to the present invention utilize fluorescence quenching to locate and/or control the thermal footprint of delivered energy, especially close to the surface area of tissue heated by a surgical energy device such as device that emits a surgical laser beam which itself is not visible to the unaided human eye, such as one or more laser beams generated by a CO₂ laser. There are a number of techniques according to the present invention to alter system settings to change the thermal footprint of delivered energy. Modifying the thermal footprint by adjusting one or more parameters of at least one delivered fluid is discussed in more detail below.

The following terms are defined as used herein. “Visible” means perceptible directly by the unaided human eye, whether viewed in line-of-sight or through an endoscope or other visualization system. “Spot size” is the radius at which the electromagnetic field amplitude and intensity drop to 1/e and 1/e² of their axial values, respectively, where “e” is the base of the natural logarithm, having a value of approximately 2.71828. “Power settings” means at least one of power level, pulse rate, pulse width, pulse shape and duty cycle for delivery of electromagnetic radiation. “Pulse shape” includes the shape of the pulse during the entire of the period of the pulse, including the time there is no energy until a subsequent pulse begins, that is, the overall shape during a single period. “Fluid control settings” means at least one of fluid flow rate or fluid temperature. A fluid control setting is increased by increasing fluid flow and/or by decreasing temperature of the fluid. “System control settings” means at least one of power settings, amount of converging or diverging of a surgical energy beam as delivered to tissue, distance of the distal end of the surgical energy delivery device from tissue, fluid control settings, and/or of fluorescence pump wavelength and/or other illuminating wavelength, distinct from any surgical wavelengths generated by the surgical energy device. “Thermal footprint” of delivered energy means the area or volume of tissue directly affected by the delivered energy both laterally, that is, in surface area, and in depth, that is, in volume, to the degree that tissue becomes thermally altered, especially when it becomes denatured or necrotic. “Thermal profile” of delivered energy means the cross-sectional boundary in three dimensions of thermal alteration within tissue, also referred to as thermal spread.

Surgical techniques employing energy, such as electro-cautery or laser cutting, produce more heat relative to cold steel. It is known that heat quenches fluorescence. Thus, to take full advantage of promising fluorescence-guided surgery, it will be important to assess and control the thermal as well as the optical tissue interactions. Visible or otherwise detectable quenching may be utilized according to the present invention as a real time temperature indicator as the surgeon nears sensitive areas, allowing for real time location, power and cooling adjustments. Analysis of the pattern of quenching may also be used to sharpen or otherwise shape or adjust thermal profiles, thereby increasing the precision of laser surgery procedures. For CO₂ laser surgery, and for surgery with wavelengths which are not visible to the naked eye, quenching may also be used to i) help locate otherwise invisible laser beams and/or ii) create a registration for subsequent procedures.

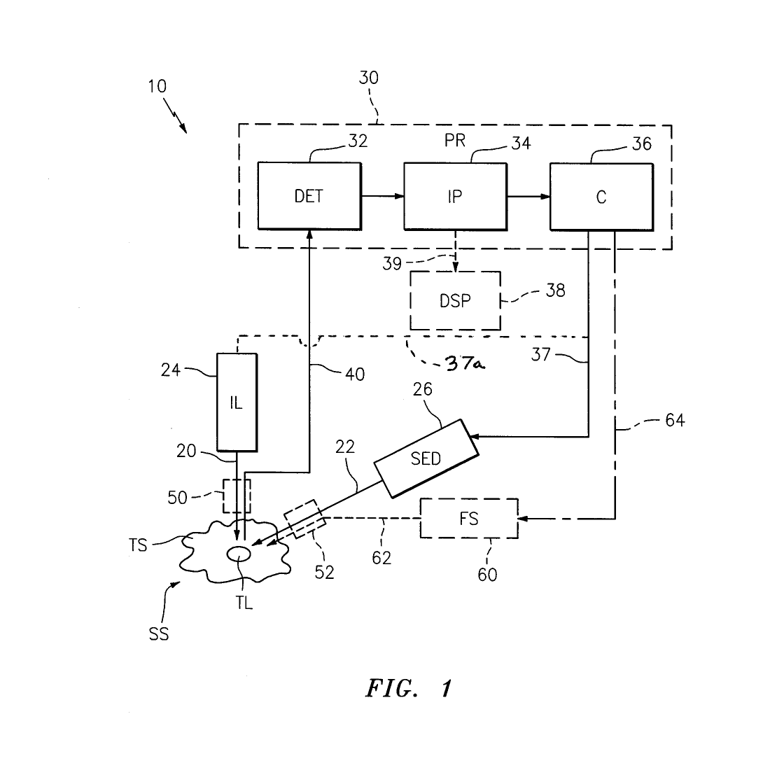

System 10, FIG. 1, has a radiation source IL, represented by box 24, of at least a first wavelength of illuminating radiation 20 directable to a target location TL on tissue TS of a patient at a surgical site SS to cause at least one type of fluorophore associated with the tissue TS to fluoresce. In certain constructions, the radiation source IL generates at least two “pump” wavelengths of radiation to stimulate fluorescence and at least one viewing or imaging wavelength of radiation to create a reflected image that can be observed by a surgeon or other user of the system. In some constructions, the radiation source IL includes a processor to control power, wavelength and/or other parameters of the illuminating radiation 20. The system 10 further includes an energy source SED, box 26, of at least a first surgical energy 22 directable to the target location TL and having a plurality of power settings. At least one of the power settings is capable of quenching fluorescence of the fluorophore at the target location TL. The system 10 preferably includes a processor PR, represented by dashed line 30, capable of adjusting at least one of a plurality of system control settings to alter the amount of quenching of the fluorophore. As described in more detail below, the system control settings include the plurality of power settings and may also include one or more fluid control settings and/or one or more aspects of the illuminating radiation.

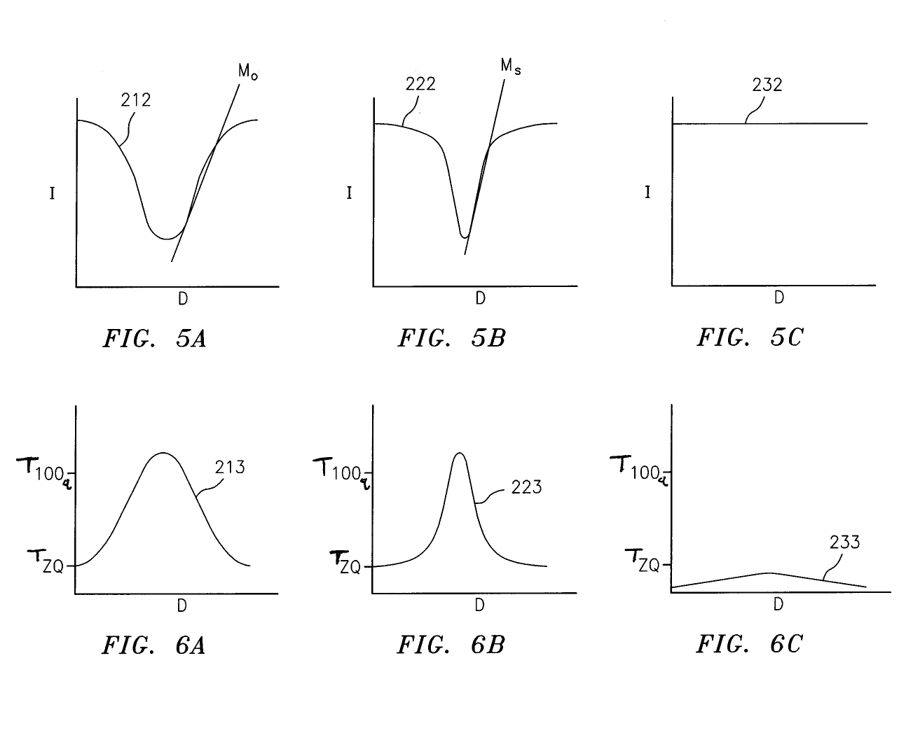

In this construction, processor PR includes a detector DET, box 32, an image processor IP, box 34, and a controller C, box 36, which communicates with surgical energy device SED as illustrated by line 37. In certain constructions, the controller C also communicates with a processor in radiation source IL as indicated by dashed line 37a to alter power, wavelength or other parameter of the illuminating radiation 20. In some constructions, processor PR includes, or communicates with, a display DSP, dashed box 38, which is visible to the surgeon or other user of system 10. Line 37 and dashed line 39 represent either cable or wireless communication with device SED and display DSP, which can be a fixed or a portable display device. Types of displayed images are discussed in more detail below in relation to FIGS. 4A-4C.

Radiation reflected (including scattered radiation) or otherwise emitted from the target location TL is directed to detector DET as represented by line 40. Dashed lines 50 and 52 represent optional access devices such as trocars. A trocar typically includes a cannula, with a proximal seal housing, that is temporarily placed through an incision in skin and underlying muscle; one or more instruments are passed through the trocar to conduct minimally invasive surgery within a body cavity. In other procedures, one or more natural body orifices are utilized. Other tools, such as retractors, suction, irrigation, or tissue manipulation or removal devices may be introduced through the same or other incisions or orifices by humans or by robotic surgical systems.

Examples of known robotic surgical systems utilizing lasers and other instruments are provided by Mohr in U.S. Patent Publication No. 2009/0171372, by Williams et al. in U.S. Patent Publication No. 2009/0248041 and by Prisco et al. in U.S. Patent Publication No. 2010/0249507, for example, all assigned to Intuitive Surgical Operations, Inc. and/or Intuitive Surgical, Inc. of Sunnyvale, Calif., which provides the Da Vinci™ robotic platform. Robotically assisted surgery through a single port utilizing an image capturing device and multiple surgical tools is described by Mohr in U.S. Pat. No. 8,517,933.

In some constructions, detector DET, box 32 in FIG. 1, includes a charge-coupled device array. Detector 32 typically is sensitive to at least one or more selected fluorescence emissions, which typically occur at a wavelength that is different from the selected fluorophore excitation wavelength. Further, as described in more detail below, it is preferred that detector 32 is sensitive to changes in intensity of emissions due to (i) quenching according to the present invention, (ii) localized concentrations of the selected fluorophores, and/or (iii) changes in intensity of the illuminating excitation radiation 20. Detector 32 and image processor IP produce individual “still” camera-type images in some constructions and, in other constructions, provide continuous video images. Optionally, light from the tissue may be directed to a detector which is sensitive to the wavelength of the surgical energy device SED. For example, see “http://www.boselec.com/products/documents/PVM-series.pdf”.

Controller C adjusts at least one of a plurality of system control settings to alter the amount of quenching of at least one type of fluorophore at target location TL. System control setting include power settings such as at least one of power level, pulse rate, pulse width, pulse shape and duty cycle for delivery of electromagnetic radiation by surgical energy device SED. In some constructions, line 22 represents at least one waveguide which passes through a handpiece such as disclosed by Temelkuran et al. in U.S. Pat. No. 7,331,954.

In certain constructions, at least one fluid source FS, box 60 shown in phantom, delivers at least one gas or liquid to target location TL as indicated by dashed line 62, as controlled by controller C through communications line 64. Several algorithms for operating system 100 are described below.

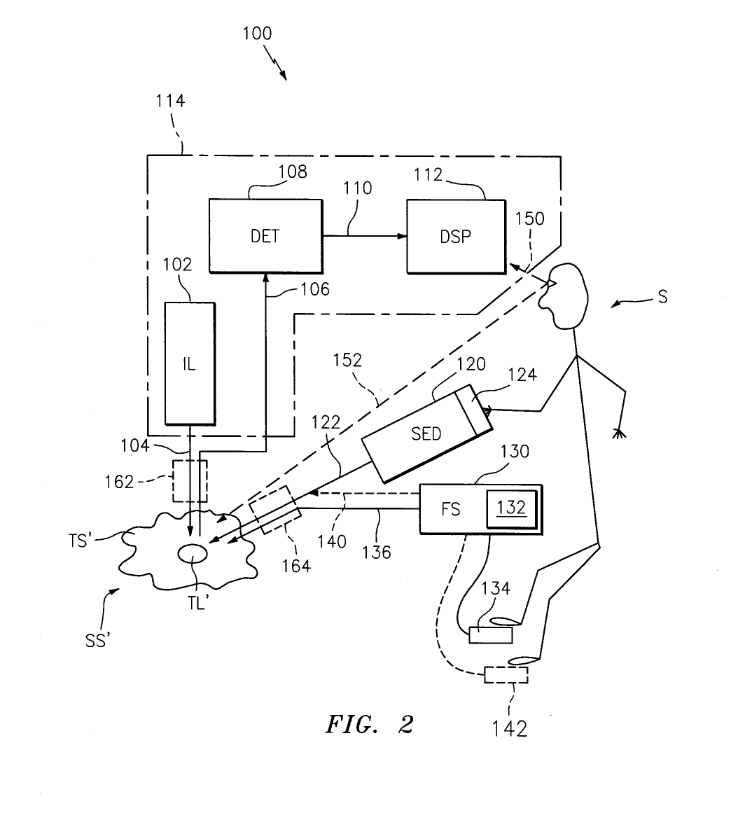

System 100, FIG. 2, includes a radiation source IL, box 102, which directs radiation 104 to target location TL′ on tissue TS' at surgical site SS' to excite at least one type of endogenous or exogenous fluorophore associated with the tissue TS. In some constructions, radiation source IL, box 102, also emits visible or other radiation to illuminate the surgical site for visualization by the surgeon. Radiation emitted and/or reflected from target location TL′ is directed, line 106, to detector DET, box 108, which includes image processing in some constructions to function as a multi-wavelength camera. Additional processing and control is provided by box 108 in some constructions. Detector 108 communicates, line 110, with display DSP, box 112, that is viewed by a user S such as a surgeon. In one implementation, radiation source 102, detector/processor/controller 108 and display 112 are part of a fluorescence endoscopy system 114, such as the one described by Cline et al. in U.S. Pat. No. 6,821,245, assigned to Novadaq Technologies of Mississauga, Ontario, Canada and Bonita Springs, Fla., US. Novadaq makes an endoscopic embodiment of the system, sold as the PINPOINT Endoscopic Fluorescence Imaging System. A similar fluorescence imaging system is also commercially available from Novadaq, sold as the Firefly™ system, for use with the Da Vinci™ robotic platform available from Intuitive Surgical.

These Novadaq endoscopy systems include a multi-wavelength light source 102, used to both pump a fluorophore present at the surgical site SS' and to illuminate the surgical site SS' and produce a reflected image ultimately depicted on display 112. A multi-wavelength camera 108 includes detectors which are sensitive to both fluorescent light and to the reflected light, which is typically visible to the unaided human eye, although such reflected light is not visible to the unaided human eye in other constructions. The fluorescence endoscopy system also includes an optic, such as a dichroic mirror, or other selective wavelength selective filter or beam splitter for splitting out the reflected light and the fluorescence. It typically also includes a processor within system 114 and a display 112 such as a video screen to display both the reflected and fluorescent wavelengths either in overlay mode, or sequentially, first the image of reflected light, and then the fluorescent image.

System 100 includes a source SED, box 120, of surgical energy which is directed, line 122, to target location TL′. In some constructions, source 120 is part of a laser surgery system such as the Intelliguide™ Laser System commercially available from OmniGuide, Inc. of Cambridge, Mass. The laser surgery system 120 includes a human interface 124, including buttons, dials, knobs or a touch screen, which can be used to select the laser power settings in some constructions. System 100 optionally includes at least one fluid source 130, which generates first fluid flow 136 and is part of the laser surgical system in some constructions. At least one fluid flow valve, represented by foot pedal control 134, also referred to as a footswitch, and a chiller/heater 132 are provided for the fluid source FS in this construction. It may also optionally include a second fluid source with its own independent fluid flow valve and chiller/heater, all represented by dashed line 140 and second foot pedal control 142. In other constructions, knobs, buttons or touch screen control replaces one or both of foot controls 134 and 142. Surgical energy source 120, fluid source 130 and associated controls are integrated into a single unit in some constructions. When the energy source 120 includes a laser and is part of a laser surgery system, the laser surgery system may also includes a flexible waveguide for delivering laser radiation 122 to the surgical site such as the BeamPath™ flexible fiber waveguides available from OmniGuide, Inc. The waveguide is hollow in some constructions. An articulated arm may also be used to deliver free space laser beams to the surgical site. One source of fluid may directed to the center of the hollow waveguide and a second source to an annular area surrounding the waveguide, as will be described further herein.

The fluorescence guided laser surgery system 100 may also include a processor such as described above for system 10, FIG. 1, whether implemented in software, hardware, or a combination of hardware and software, for analyzing the fluorescent images and using the analysis to control the laser system including the amount of laser energy (e.g. by power setting or duration of energy application) and fluid flow settings. The processor selects and sets some or all of the laser system settings in some constructions and, in other constructions, the surgeon S selects and sets some or all of the laser system and/or fluid control settings as represented by user viewing line 150 from surgeon S to display 112, especially when minimally invasive surgery is performed through access devices 162 and 164, and by alternative viewing line 152 from surgeon S directly to surgical site SS′, especially when open surgery is performed. The processor may automatically select, or a surgeon may manually select, one or more settings on illumination source IL which, in some constructions, is used both to excite the fluorophores and to illuminate the surgical site to enhance visibility and viewing by the surgeon.

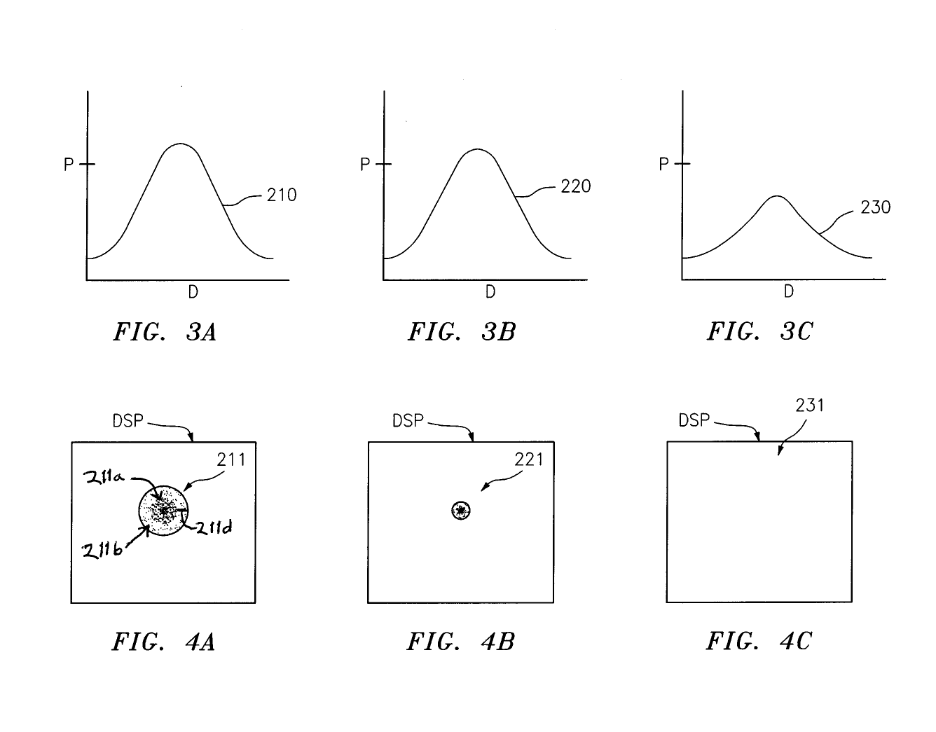

Another aspect of the invention is to use fluorescence quenching to determine and sharpen the thermal profile at the laser tissue interface, such as by delivering cooling fluid around the laser spot at the laser-tissue interface, and thereby sharpening the thermal profile at that interface. FIGS. 3A-3C schematically represent cross-sections of energy level delivered to tissue under a first normal setting, certain profile-sharpening settings, and an insufficient-to-quench power setting, respectively. Fluorescence relies on at least one radiative transfer of an electron from an excited state, and heat increases the probability of non-radiative transfers, therefore heat reduces fluorescence intensity.

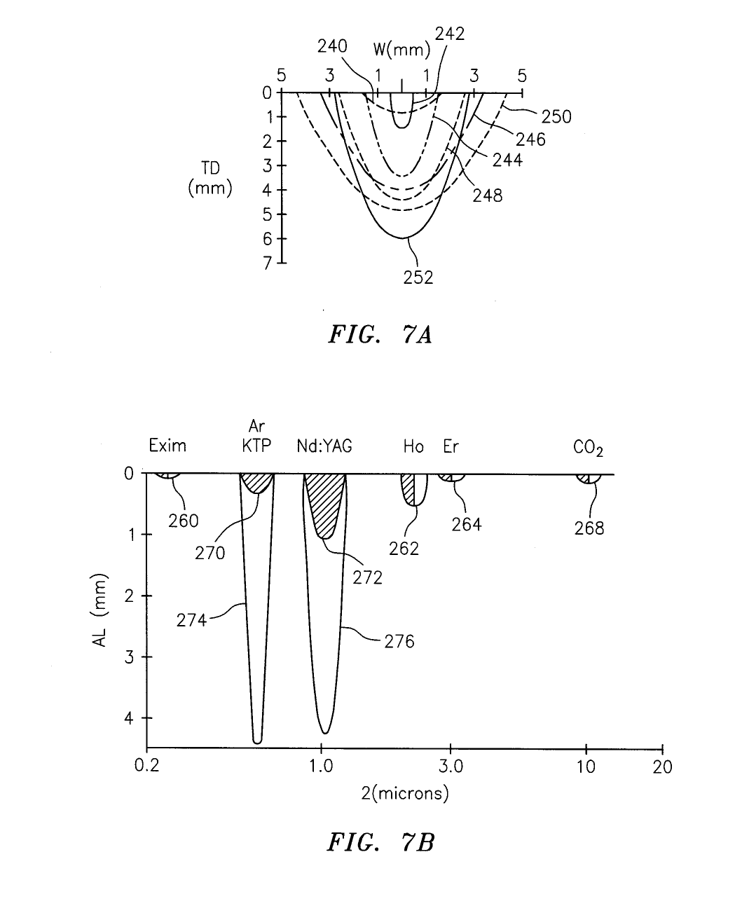

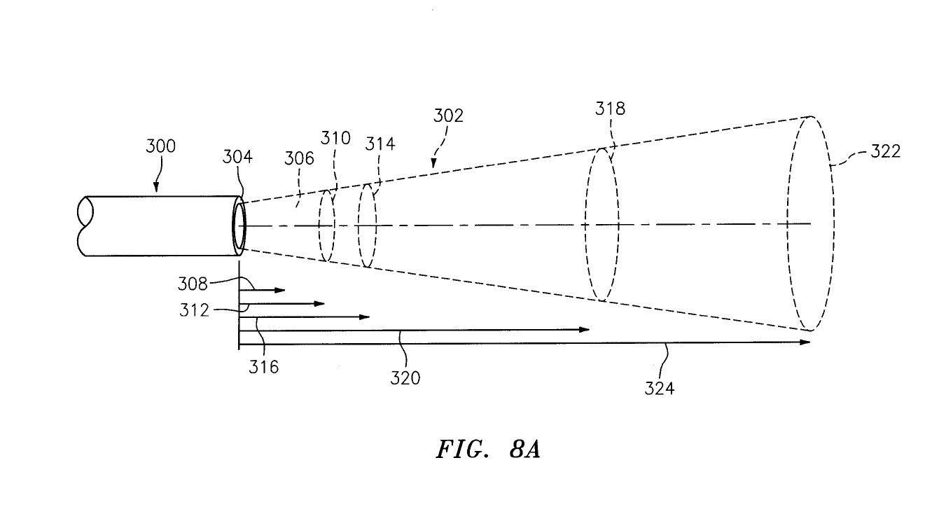

More particularly, FIG. 3A schematically shows a plot of the energy distribution in a Gaussian laser beam as curve 210. Assuming CW (continuous wave) operation, and absent intervening optics, this also approximates the energy distribution at the laser tissue interface. The resulting dark spot 211, FIG. 4A, displayed on video screen DSP results from unmodified laser quenching of fluorescent tissue. It is shown schematically as a central region 211a where most or all the fluorescence is quenched, surrounded by a region 211b, which may appear to be toroidal in cross-section, where some of the fluorescence is quenched. It is expected that region 211b will exhibit graded quenching, from almost entirely quenched, to almost not noticeably quenched at the outer periphery of region 211. Line 211d indicates the width of the region 211b where some of the fluorescence has been quenched. Note that there could be no region where all of the fluorescence is quenched, i.e. no 211a (not illustrated), and also no region where even some of the fluorescence is quenched, i.e. no 211b either, which is illustrated in FIG. 4C. Once the system has been calibrated, the fluorescence intensity may be used to assess tissue temperature and/or damage. There is a corresponding schematic fluorescence intensity of curve 212, FIG. 5A, and the corresponding schematic temperature profile curve 213, FIG. 6A, for the curve 210, FIG. 3A. If total quenching occurs, then the lowest portion of curve 212 would appear as flattened at zero intensity over the region of total quenching. In FIG. 6A, Tzq is the temperature at which there is no detectable quenching for steady state illumination of the pump light, and T_{100q} is the temperature at which there is no detectable fluorescence. At and above this temperature, there are no radiative transitions, only non-radiative ones. Thus, fluorescence is observable only below T_{100q}. Note that above T_{100q}, the lack of fluorescence does not necessarily correspond to a particular tissue temperature, because the tissue temperature could be at a range of temperatures all above T_{100q}.

The maximum slope mₒ of the graph of fluorescence intensity 212 in FIG. 5A shows one measure of sharpness of the thermal profile. An alternate measure is the distance at the tissue interface between no detected quenching and maximum detected quenching, illustrated as distance 211d in FIG. 4A with a correspondingly smaller distance in FIG. 4B. The sharper profile has a steeper slope and smaller transition region. Since laser cutting is accomplished by ablating a line of tissue via boiling or vaporization of water in the tissue, an idealized temperature profile of a very precise cut would have normal body temperature on either side of the cut, a rapid spatial transition to the ablation temperature and a rapid transition back to normal temperature, permitting a desirable amount of hemostasis and coagulation at the edge of the cut. Sharper thermal profiles result in more precise cuts. Less sharp profiles may result in unwanted tissue damage and or coagulation or denaturing at the edge of the cut.

The laser beam is heating a volume of tissue, having a parabolic shape in cross section, not only a spot on the surface of the tissue. The size of the spot, however, may be taken as an indication of the depth of the heating as well as its lateral extent, with depth indication being useful to surgeons. Using a pulsed laser source is expected to reduce the depth of heating as well as the lateral extent, while an annular cooling fluid approach is expected to modulate temperature primarily at the tissue-laser interface.

There is more than one approach to narrowing a temperature profile. One approach is to set the energy pulse intensity, width and rate such that the total time the tissue is exposed to laser energy is less than the thermal relaxation time of the tissue being ablated. Since heat diffuses from an area in proportion to its perimeter, more heat will diffuse from the edges of the spot than from the center which will help preferentially to heat the middle of the spot relative to its edges.

Another approach is to make the energy spot size smaller by focusing optical energy using lenses or by controlling the distance between tissue and the energy exit point, such as when an optical fiber is used to guide energy to the tissue. Certain techniques for altering the energy spot size utilizing optical components in a handpiece are provided by Shurgalin et al. in U.S. Patent Publication No. 2013/0064515.

Yet another approach is to direct cooling fluid preferentially around the spot relative to its center, as illustrated in FIG. 3B where laser power P distribution striking the laser-tissue interface, curve 220, is the same as curve 210, FIG. 3A. Directing cooling fluid to the laser-tissue interface and/or certain other approaches to thermal profile sharpening as taught herein will result in fluorescence profile sharpening as represented by smaller quenched spot 221, FIG. 4B, a smaller transition region, and/or a steeper, narrower curve 222, FIG. 5B. The maximum slope mₛ of the fluorescence intensity versus distance curve 222 has increased for approaches according to the present invention utilizing the same laser power settings so that slope mₛ is greater than slope mₒ of FIG. 5A. Similarly, thermal profile curve 223, FIG. 6B, is narrower that curve 213, FIG. 6A.

The cross sections of fluorescence intensity and temperature shown schematically by curve 222, FIG. 5B, and curve 223, FIG. 6B, respectively, are not necessarily Gaussian in shape. It is also to be noted that the shape of the curves in FIGS. 3B, 5B and 6B are schematic approximations and will vary depending on changes to various parameters controlled according to the present invention.

For example, when a Gaussian beam exits from a waveguide coupled to a laser operating in continuous wave (“CW”) mode, and there are no further optics at the distal end of the waveguide, and the invention is practiced using annular fluid flow, then curve 220, FIG. 3B, is substantially the same as curve 210, FIG. 3A. Thermal footprint control is achieved via controlling at least one of fluid flow rate and fluid temperature in this example.

Under different conditions, when a Gaussian beam exiting from a waveguide coupled to a laser operating in CW mode has been collimated and/or focused to reduce the beam spot size before and also at the laser tissue interface, and if the power settings are unchanged relative to those represented by FIG. 3A then, for a lossless collimating and/or focusing optic, the area under the intensity curves in FIG. 3A and a hypothetical revised 3B is the same. However, the overall energy density at the laser tissue interface will increase, and curve 220 becomes taller and narrower than curve 210. Thermal footprint control is practiced via at least one of selection, control, and placement of the collimating and/or focusing optics in this example.

Under other conditions, curve 220, in another hypothetical revised FIG. 3B, can be redrawn to represent a time averaged intensity profile of a pulsed Gaussian beam exiting from a waveguide, coupled to a laser with a 50% duty cycle, having no further optics at the distal end of the waveguide. If the peak power settings and beam shape are unchanged relative to those represented by curve 210, FIG. 3A, then the area under curve 220, of the hypothetical revised FIG. 3B, will be approximately half of the area under curve 210, FIG. 3A, with curve 220 being approximately one-half as tall and approximately the same width as curve 210. Thermal footprint control is practiced via control of at least one of pulse frequency and pulse shape in this example.

Curve 230, FIG. 3C, and corresponding “blank” display screen 231, FIG. 4C, uniform fluorescence curve 232, FIG. 5C, and relatively flat curve 233, FIG. 6C, illustrate directing a low power surgical beam toward the surgical site, such that there no observable decrease in fluorescence. Any temperature increase therefore, is below a temperature which could cause quenching given the steady state pumping conditions. Thermal properties vary with tissue types, and quenching may be reversible with cooling and also irreversible. When it is irreversible it is regarded as a sign that the tissue has started denaturing or otherwise changing molecular structure. Thus, the laser system settings at which fluorescence quenching is first detected and reversible, and first produced irreversibly may be used to characterize tissue and also to select settings for subsequent procedures.

Brian Masters observed in his Master's Thesis an average decrease in endogenous fluorescence intensity of approximately 1% for every increase in temperature of 1° C. Thus, a ten degree increase in temperature would result in a 10% decrease in fluorescence, and a twenty degree increase in a 20% decrease in fluorescence. See “Variation of Fluorescence With Temperature in Human Tissue”, by Daniel Barton Masters, submitted in partial fulfillment of the requirements for the degree of Master of Science in Biomedical Engineering, May, 2010 Vanderbilt University.

Observed quenching occurs at temperatures well below the temperature at which water vaporizes, 100° C., and thus well below the temperature at which ablation occurs. Most tissue is approximately 70 percent to 80 percent water. Thus, it is a realization of the present inventors that such quenching can make the “invisible” energy beams, such as a CO₂ laser beam, effectively visible before it delivers energy used for cutting and ablation.

The relationship between tissue temperature and tissue change, for both tissue effect and visual effect, is shown in Table I:

TABLE I

| TEMPERATURE | TISSUE EFFECT | VISUAL EFFECT |
|---|---|---|
| 37° C.-60° C. | Heating | No Change |
| 60° C.-90° C. | Denaturation/Onset of | White/Grey |
| | Coagulation | |
| 90° C.-100° C. | Drying/Puckering | Wrinkling/ |
| | | Puckering |
| 100°+ C. | Vaporization (Cutting/Ablation) | Golden/Char/Smoke |

When water in tissue reaches 100° C., water vapor and solid particulates are created, which appears as “smoke”. Whether cutting or ablation occurs depends on several system control settings as described in more detail below.

Denaturing interferes with fluorescence. As surgical procedures strive to mark not only tissue to be removed, but also mark without damaging protected tissue, controlling heat profiles becomes increasingly important.

Returning to surgery utilizing systems and methods according to the present invention, and using a decrease of fluorescence of 1% per degree centigrade, a body temperature of 37° C., an estimate that 50° C. is the temperature below which the tissue generally fully recovers its body temperature fluorescence intensity, and an estimate that 70° C. is the temperature above which tissue generally does not recover its body temperature fluorescence intensity, it can be seen that, first: if a decrease in intensity of about 13% can be detected, either by the unaided eye of the surgeon, or by a sensor, (50° C.−37° C.=13° C., resulting in a reversible fluorescence intensity decrease of approximately 13%), then confirmation of the location of a surgical beam can be accomplished with no irreversible tissue alterations after the beam is turned off Additionally, the thermal footprint of the energy can be visualized during tissue removal (e.g. cutting or ablation) before irreversible tissue changes occur.

Second, if a change of 40% can be detected, either by the unaided eye of the surgeon, or by a sensor, (77° C.−37° C.=40° C., resulting in a fluorescence intensity decrease of approximately 40%), i) confirmation of the location of a surgical beam can be accomplished, and ii) if desired, a registration mark can be made to identify structures for reference during the procedure. According to this analysis, using 100° C. as the temperature at which cutting and ablation starts, up to a 63% reduction in fluorescence intensity can be generated and detected before cutting into tissue.

The quenching may also differ between tissue types, as appears to be suggested by the work of Daniel Barton Masters. It is known that early stages of cancer may suppress certain emission bands of fluorescence in tissue, as stated by Cline et al. in U.S. Pat. No. 6,821,245, for example. Once calibrated and characterized according to the present invention, differences in quenching are expected by the inventors to provide further assistance in differentiating between such tissue types.

Desirable characteristics of such fluorophores are i) biocompatibility, and preferably FDA approval, such as ICG (indocyanine green), ii) a noticeable change in intensity before a temperature increase to 100° C., and thus before cutting and ablation occur. In an embodiment of the present invention, a noticeable and reversible change in intensity would occur before coagulation and protein denaturation occurs, as in the Master's example of endogenous fluorophores. In another embodiment, an irreversible decrease in fluorescence occurs before cutting and ablation occurs.

Fluorescent lifetime versus temperature has been studied for a family of often-used exogenous dyes, the boron dipyromethane dyes, or BODIPY dyes (4,4-Difluoro-5-Phenyl-4-Bora-3a,4a-Diaza-s-Indacene-3-Propionic Acid, Succinimidyl Ester). Shi and Parks studied BODIPY-R6Gⁱ covalently bonded to a variety of peptides as described in “Fluorescence Lifetime Probe of Biomolecular Conformations” by Xiangguo Shi and Joel J. Parks, J Am Soc Mass Spectrometry 21, 707-718 (2010). Their data show that fluorescent lifetimes can be engineered via selection of the molecule covalently bonded to the starting dye. The data also show that for their system, a factor of two change in quenching rate is achievable over the temperature range of interest, from approximately 310° K, which is body temperature, to 373° K, the temperature at which water boils and ablation and cutting occur. The absolute intensity and change in intensity will be a function of the concentration of the fluorophores and quenching centers, per the Stern-Volmer equation:

F₀/Fₘ=1+κ_{q}τ₀[Q] (1)

where Fₘ is the measured fluorescence with quenching, F₀ is the initial, unquenched fluorescence, κ_{q}, is the quencher rate coefficient, τ₀ is the lifetime of the radiative excited state without the quencher present, and [Q] is the quencher concentration. Their success in readily modulating quenching rates by derivatizing a well-known fluorophore suggests to the present inventors that a similar approach could readily be used to customize an exogenous dye useful for the present invention.

Sharpening the thermal profile may be accomplished by increasing the flow rate of cooling fluid surrounding the center of the spot or decreasing its temperature. Apparatus for delivering fluid to cool a waveguide is disclosed in U.S. Pat. No. 7,331,954 by Temelkuran et al., for example. See also U.S. Pat. No. 6,343,174 by Neuberger for one or more fluid delivery channels for other purposes.

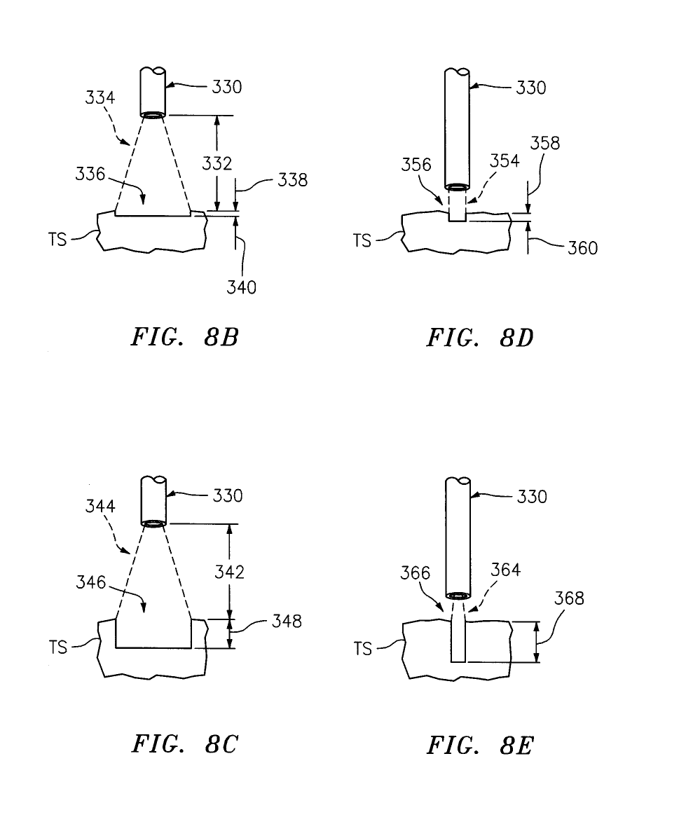

Most common sources of energy used in surgery (e.g. electro-cautery, laser and ultrasonic energy) have a thermal footprint associated with them. FIG. 7A is a chart of thermal footprints, also known as thermal spread, showing width W and depth TD, both in millimeters, of thermal damage to tissue by various surgical energy devices utilizing different energy sources. In order of increasing depth TD, curve 240 is a bipolar RF profile, curve 242 is a CO₂ laser profile, curve 244 is a profile of a Plasma Knife™ device available from Gyrus ACMI, curve 246 a profile of a Thulium laser, curve 248 is a profile of a Coblation™ RF device from Arthrocare Corp., curve 250 is a monopolar RF profile, and curve 252 is a profile of a Harmonic Scapel™ device from Ethicon Endo-Surgery, a division of Johnson & Johnson, which utilizes both ultrasonic and RF energy. Note that the width W is not necessarily related to the depth TD of thermal damage. For example, the narrowest width W is provided by the thermal footprint of a CO₂ laser, curve 242, although the shallowest depth TD is provided by a bipolar RF device, curve 240. One can see that each thermal footprint spreads both laterally and in depth to varying degrees based generally on the type of surgical energy. Minimizing thermal footprint according to the present invention enables more tissue sparing procedures and helps to avoid damage of critical structures, such as nerves and/or blood vessels, during surgery.

Laser surgery typically utilizes long, thin, flexible solid or hollow waveguides to deliver specific wavelengths of electromagnetic radiation. Solid core silica fibers, for example, are utilized to guide wavelength of KPT (532 nm), Nd:YAG (1.06 μm), Ho:YAG (2.1 μm) and Tm: YAG (2 μm) lasers for various medical applications. For CO₂ laser beams (approximately 10.6 μm wavelength), hollow waveguides are useful, as the CO₂ wavelength is generally highly absorbed in materials traditionally used for optical fibers, such as silicates and thermoplastic polymers. A high omnidirectional reflector is disclosed in U.S. Pat. No. 6,130,780 to Joannopoulos et al.

FIG. 7B is a chart of penetration and absorption of various wavelengths of laser energy to absorption lengths AL in pigmented and unpigmented tissue, with the x-axis ranging from 0.2 microns to 20 microns on a logarithmic scale and the absorption length AL in millimeters on a linear scale along the y-axis. The effect on pigmented tissue is depicted with cross-hatching. For example, profiles 260, 262, 264 and 268 for Excimer, Ho, Er and CO2 lasers, respectively, are approximately the same for both types of tissue. In contrast, Argon KTP and Nd:YAG lasers have relatively shallow profiles of 270 and 272 for pigmented tissue and much deeper absorption profiles 274 and 276, respectively.

Flexible hollow waveguides are manufactured in some techniques by drawing structured thermoplastic preforms. Examples of such a structure are described by Harrington et al. in U.S. Pat. No. 5,440,664 and by Fink et al. in U.S. Pat. Nos. 6,463,200 and 7,311,962, in which a dielectric stack of materials having different refractive indices is arranged in concentric cylinders about the waveguide axis thus providing the mirror structure that guides the radiation. Flexible hollow waveguides drawn from structured thermoplastic preforms are also disclosed in U.S. Pat. No. 7,272,285 to Benoit et al. and U.S. Pat. No. 7,295,734 to Bayindir et al., as well as in the following U.S. patents assigned to OmniGuide, Inc.: U.S. Pat. No. 6,788,864 by Ahmad et al.; U.S. Pat. No. 6,801,698 by King et al.; U.S. Pat. No. 6,898,359 by Soljacic et al.; and U.S. Pat. No. 7,142,756 by Anderson et al.

At times, certain surgical uses of energy delivery devices such as waveguides may result in tissue debris, fluid, or smoke being generated. Such tissue debris may absorb delivered energy, including backscattered laser energy, and heat or otherwise interfere with the waveguide. Such tissue debris may impede or slow normal passive cooling resulting from thermal dissipation, and/or impede more active cooling resulting from delivered fluid, including gas flow through the waveguide core. The combination of increased heating and reduced cooling may overheat and thus damage the waveguide.

One approach to protect the portion of the surgical energy device is to flow fluid through a conduit such as hollow core waveguides. Gas flow may be used for clearing tissue debris and blood during tissue cutting, for cooling the waveguide and for therapeutic reasons such as assisting tissue coagulation. The gas flowing out of the waveguide may also assist in keeping the waveguide core from clogging and from damage due to the splattering, splashing, or deposition of tissue debris, including smoke and fluids. Protection of the waveguide distal end may also be achieved by a tip attached to the waveguide distal end, such as disclosed by Temelkuran et al. in U.S. Pat. Nos. 7,167,622 and 7,331,954, by Goell et al. in U.S. Pat. No. 8,280,212, and by Anastassiou et al. in U.S. Patent Publication No. 2014/0088577, all assigned to OmniGuide, Inc. of Cambridge, Mass.

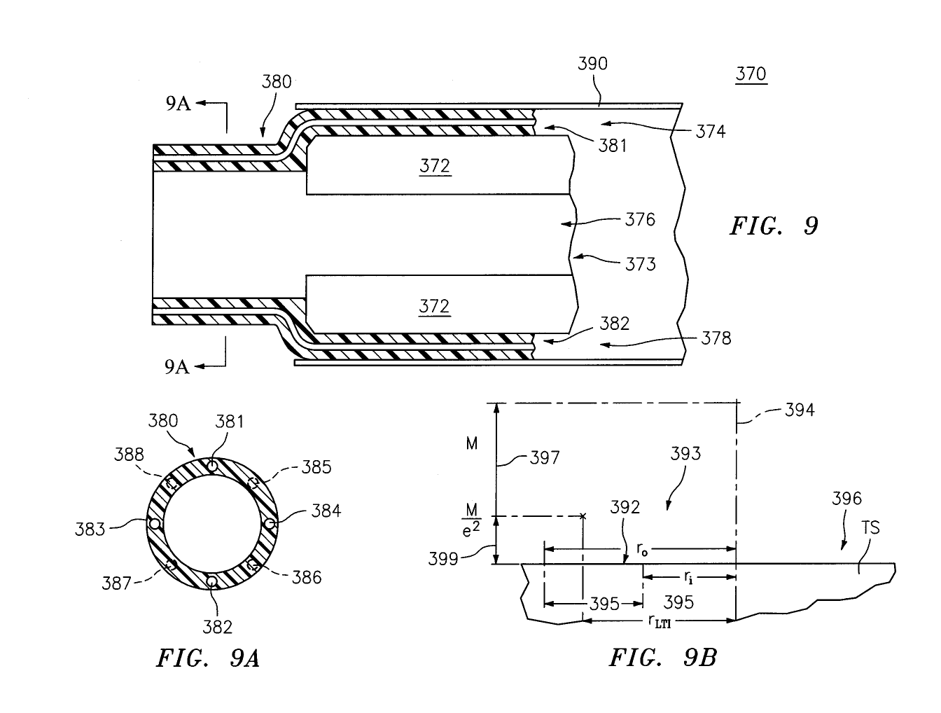

FIG. 8A illustrates a waveguide 300 for optical radiation including an opto-mechanical system to control the spot size of energy applied to the tissue for a diverging energy beam 302 coming out of the waveguide 300. One example is a hollow core fiber currently available from OmniGuide, Inc. that has an inner diameter of approximately 320 microns In one construction, the spot diameter at distal tip 304 is 320 microns, the spot diameter 306 at distance 308 of 1 mm is 400 microns, the spot diameter 310 at distance 312 of 2 mm is 485 microns, the spot diameter 314 at distance 316 of 3 mm is 570 microns, the spot diameter 318 at distance 320 of 2 cm is 2.0 mm, and the spot diameter 322 at distance 324 of 3 cm is 2.8 mm.

In one construction, a waveguide tip with a variable cantilevered distal end portion length allows a user to select a spot size. The illustration of the diverging cone 302 of radiation emerging from the waveguide 300 illustrates how selecting the stand-off distance determines the spot size. The spot size may be determined approximately by directing the laser at a wooden tongue depressor and observing charring of the wood, for example. The spot size for a given waveguide beam divergence may be set during manufacturing, after the product has been sold but before surgery, by the surgical staff, or after or during a procedure. It may be possible to set the stand-off distance once, or many times. The stand-off distance may be set using a push or pull mechanism in the conduit.

The spot size of the laser radiation emitted from the distal tip affects the power density of the laser energy and thereby defines laser tissue interaction, such as cutting or ablation mode, as well as the rate of cutting or ablation. In general, a beam exiting a optical waveguide diverges as shown in FIG. 8A. Therefore, spot size may be controlled by setting a distance between an exit point of the laser radiation and the tissue, i.e., by control of the stand-off distance.

Another way to control the distance between waveguide and the tissue may be by using a proximity sensor built into the waveguide, jacket, or conduit. This proximity sensor may measure a distance to the tissue and provide a feedback to the user or computer interface. Distance may be controlled by the user or pre-programmed into a computer that automatically maintains a preset distance by adjusting the position of the manipulator.

FIGS. 8B and 8C are schematic illustrations of low power and higher power, respectively, with a CO₂ laser waveguide 330 spaced relatively far from tissue. At low power input of 2-4 watts and a distance 332, FIG. 8B, of 2-3 cm from tissue TS, diverging CO₂ laser beam 334 causes superficial ablation 336 having a relatively shallow depth as represented by arrows 338 and 340. By comparison, at a higher power input of 15-20 watts and a distance 342, FIG. 8C, of 3-5 cm from tissue TS, diverging CO₂ laser beam 344 causes ablation and coagulation 346 having a deeper depth as represented by arrow 348.

FIGS. 8D and 8E are schematic illustrations of low power and higher power, respectively, with a CO₂ laser waveguide 330 spaced relatively close to tissue TS at a distance of 0.2-0.3 cm in both examples. At a first power input of 6-12 watts, diverging CO₂ laser beam 354, FIG. 8D, causes fine cutting 356 having a relatively shallow depth as represented by arrows 358 and 360. By comparison, at a second, higher power input of 12-20 watts, diverging CO₂ laser beam 364 causes cutting 366 having a deeper depth as represented by arrow 368.

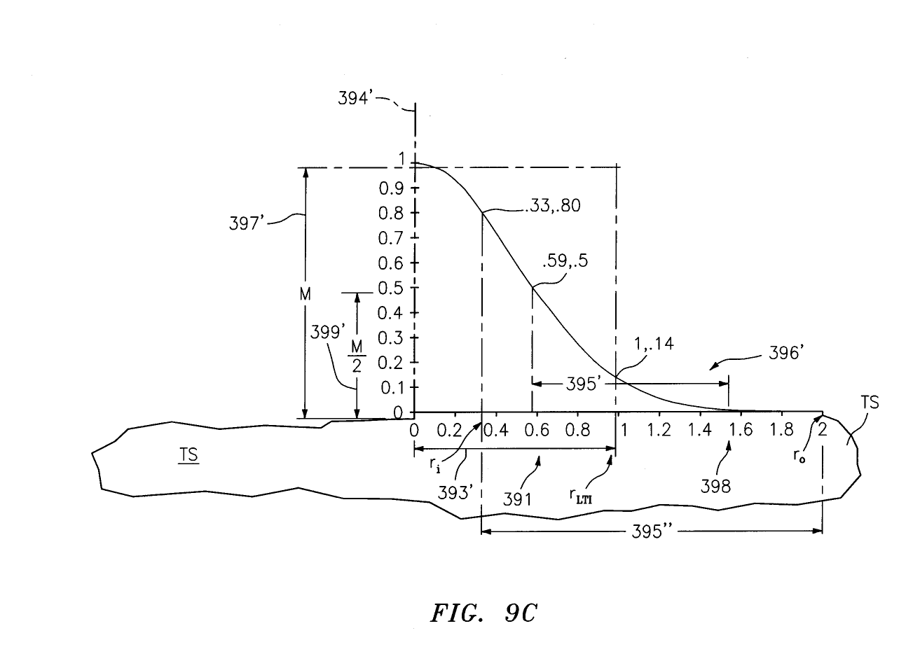

FIG. 9 is a schematic side cross-sectional view of the distal end of a surgical energy device 370 including a laser waveguide 372 capable of delivering at least one cooling fluid, represented by arrows 374, 376 and 378, to alter a thermal profile according to the present invention. The cooling fluid may be actively cooled to a selected temperature for this purpose. In this construction, distal tip 380, positioned between waveguide 372 and outer conduit 390, delivers at least one of cooling fluid 376 to the center of a laser spot, or cooling fluids 374 and 378 to a region or regions surrounding a laser spot, at the laser-tissue interface. The optical radiation is transmitted through hollow core 373 in this construction. The waveguide 372 may optionally have cooling fluid 376 flowing through the core 373. The tip 380 has four channels 381, 382, 383 and 384 in the illustrated construction, as best seen in FIG. 9A, through which cooling fluid is directed at an annular region between one-quarter to three times the spot size at the laser tissue interface, more preferably between one-third to two times the spot size, of an energy beam delivered through waveguide 372. Optionally, additional channels 385, 386, 387 and 388 are provided, as indicated in phantom in FIG. 9A. Preferably, but not necessarily, all of the fluid delivery channels 381-388 are coaxially centered with respect to the center of the spot at the target location on tissue. The spot size of a laser beam at the laser tissue interface is a function of beam divergence and the distance between the end of the waveguide and the tissue interface, such as described above in relation to FIGS. 8A-8E above.

As schematically illustrated in side view in FIG. 9B, the cooling fluid preferably is directed at an annular or toroidal region 392, having a width represented by arrow 395, located between approximately one-third and two times the radius r_{LTI} of the laser spot 393 at the laser-tissue interface 396 and centered with respect to centerline 394 of the spot. The term “r_{LTI}” is the radius at which the electromagnetic intensity drops to 1/e² of its axial or along-the-centerline intensity value, where “e” is the base of the natural logarithm, having a value of approximately 2.71828. The maximum axial or along-the-centerline value as delivered to the laser tissue interface is depicted by arrow 397 having a value “M”. The value at r_{LTI} is depicted by arrow 399 having a value M/e², with arrow 399 drawn as overlapping a portion of arrow 397 in FIG. 9B. The inner radius rᵢ of the annulus represents the distance along the laser-tissue interface measured from the centerline 394. The outer radius rₒ of the annulus represents the distance along the laser tissue interface measured from centerline 394. The cooling fluid is directed toward an annulus defined by these two radii, concentrically arranged around the center line 394. Thus distance 395 illustrates the width of the cooling annulus as being rₒ-rᵢ in this example.

Two further examples of similar ring- or toroidal-shaped cooling regions 395′ and 395″ are shown in FIG. 9C. In one example, the inner radius 391 of the annulus is approximately 0.59 times r_{LTI} and represents the distance along the laser-tissue interface where beam power is approximately one-half of maximum beam power M, represented by arrow 397′, as delivered to the laser-tissue interface. One-half M is represented by arrow 399′. The cooling fluid extends to an outer radius 398, approximately 1.52 times r_{LTI} as measured from the centerline 394′, where the power is approximately one percent of M.

A further example of an alternative, wider cooling annulus is indicated by arrow 395″ in FIG. 9C. In this example, the inner radius rᵢ of the annulus is approximately 0.33 times r_{LTI} and represents the distance along the laser-tissue interface as measured from centerline 394′ where beam power is approximately 80% of maximum beam power M. The cooling fluid extends to an outer radius rₒ, approximately two times r_{LTI} as measured from centerline 394′, where the power is negligible. Another potential choice for the inner radius is the inflection point in the Gaussian curve, where the slope of the curve changes, which is approximately 0.7 r_{LTI}, and is not shown on in FIG. 9C. These examples are for illustrative purposes only and may vary with different tip designs, tissue types, system settings, and procedures.

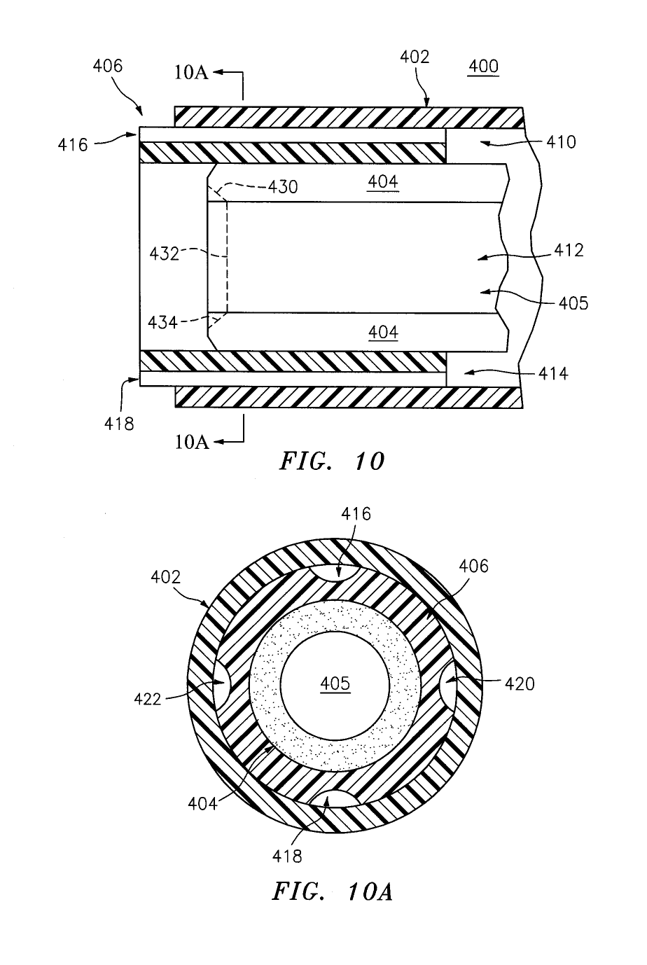

FIGS. 10 and 10A are views similar to those of FIGS. 9 and 9A of an alternative surgical energy device 400 with an outer conduit 402, an inner waveguide 404 with a hollow core 405, and a distal tip 406. All illustrated components can be fabricated from common medical device materials for catheters and waveguides. Device 400 is capable of delivering at least one cooling fluid, represented by arrows 410, 412 and 414, to alter a thermal profile according to the present invention. The optical radiation is transmitted through hollow core 405 in this construction and the waveguide 404 may optionally have cooling fluid 412 flowing through the core 405. The tip 406 defines four channels 416, 418, 420 and 422 on its exterior surface in the illustrated construction, as best seen in FIG. 10A, through which cooling fluid is directed at an annular region typically between one to four times the spot size of an energy beam delivered through waveguide 404. Dashed lines 430, 432 and 434 represent chamfers or other topographic features that are added in some constructions to alter fluid flow dynamics. In another construction, one or more channels such as grooves are cut in the interior surface of distal tip 406 such that one or more cooling fluids flow between tip 406 and waveguide 404. In yet another construction, the tip itself also serves as a waveguide, such as for optical tips disclosed by Shurgalin et al. in U.S. Patent Publication No. 2013/0064515.

As mentioned above, others have suggested utilizing one or more visible aiming beams or a physical projection such as a stand-off tip to assist locating invisible surgical energy beams. For both these pre-alignment approaches, any verification of the alignment takes place outside the patient. Therefore, it is useful and advantageous for the surgeon also be able to easily check and verify correct beam placement inside the patient before the laser power is increased to a power where cutting or ablation takes place. It is also useful for the surgeon to be able to see the actual location of the beam before it cuts or irreversibly alters the tissue, and not rely on proxies, such as the position of a second visible beam or the position of a tip. Thus, it is desirable to see the position of a low-energy beam, where the energy is too low to cut or ablate tissue, and thereby verify proper placement. Systems and methods according to the present invention enable a user to make adjustments to ensure such placement before the power is increased to a level where it causes irreversible tissue alterations.

Iteration toward desirable laser power and cooling fluid settings is accomplished manually, using the surgeon's eyes as the sensor, in certain embodiments such as those illustrated for system 100 in FIG. 2 above. In other constructions, such as shown for system 10 in FIG. 1 or in combinations of systems 10 and 100 and other alternatives within the scope of the present invention, including robotic implementations, as will be apparent to those skilled in the relevant art after reading this disclosure, one or more automated feedback loops are implemented to slowly ramp up or decrease one or more selected system control settings such the amount of cooling on a test spot to discover the settings at which fluorescence is first visibly and reversibly quenched at a surgical site, and then visibly and irreversibly quenched.

It can be also useful for the surgeon to have a visual feedback about energy thermal spread during surgical procedure while performing tissue cutting or ablation. Having this feedback may be important in helping to avoid damage of critical structures. In one procedure according to the present invention, at least one critical structure such as a nerve or blood vessel is marked with at least one type of exogenous fluorophore. Fluorescence of the critical structure is quenched as the thermal energy footprint approaches the structure. Decrease of fluorescence intensity corresponding to the threshold temperature (e.g. 50° C. if no irreversible tissue damage is desired) can be detected. The laser or other surgical energy device utilized according to the present invention can be turned off, or proper adjustment of controllable parameters such as laser power, duration or spot size can be made to avoid further energy spread into the critical structure.

As surgeons approach sensitive areas, it may be desirable to adjust laser settings so as to remove tissue more slowly, or to avoid inadvertent damage to such areas due to heat spreading. Fluorescence quenching, starting first in less sensitive areas, may be used to adjust the laser power and pulse rate and duty cycle, energy density or surrounding cooling fluid flow, to create the sharpest dark spot. This setting may then be used for the entire surgery and or as the surgery proceeds to areas closer to structures which are to be protected. The setting adjustments may be made manually, relying on the human eye as the fluorescence intensity detector, or via an electronic sensor and computer interface.

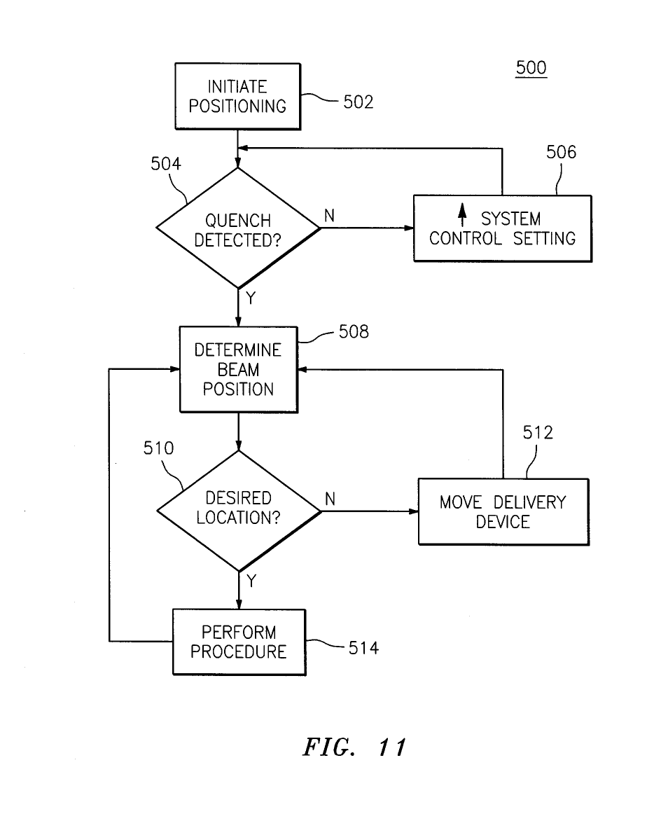

Flow chart 500, FIG. 11, illustrates steps according to the present invention, such as performed by processor 30, FIG. 1, to position delivery of surgical energy to optimize the location where it strikes tissue. The positioning algorithm is initiated, step 502, which includes activating the surgical energy device at a setting below that at which tissue damage occurs. If no quenching is detected at step 504, then at least one system control setting is increased, such as decreasing the distance of a waveguide to the target location on tissue or increasing the power level of the energy device, and the logic returns to step 504. Once quenching of fluorescence is detected, the logic proceeds to step 508 to determine if the energy beam is correctly positioned. If the location of the quenched fluorescence is not optimal, step 510, or is not otherwise customized or tailored as desired, then the delivery device such as a handpiece with a waveguide is moved slightly in a desired direction, step 512, and the logic returns to step 508. Once the desired location is achieved, the surgical procedure can be directly performed, step 514. Alternatively, one or more of optimizing spot size, thermal profile, and/or calibrating the surgical system can be conducted before the surgery is actually performed.

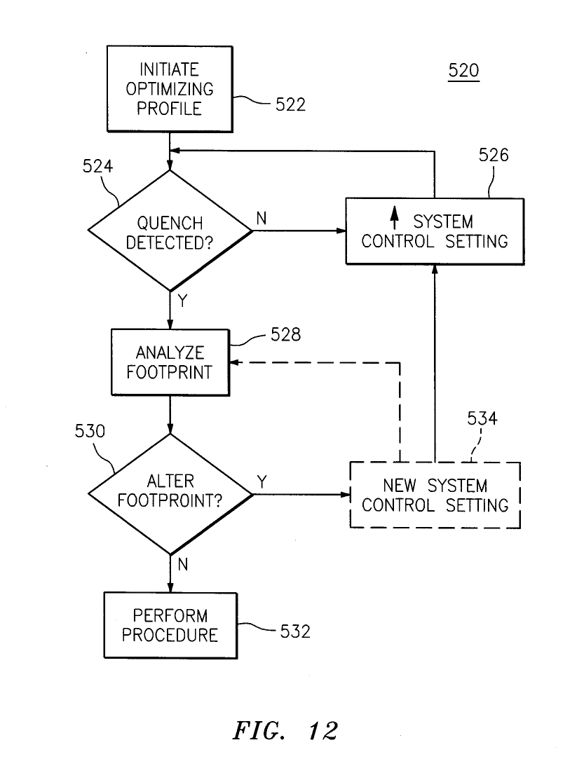

Flow chart 520, FIG. 12, shows steps to optimize the thermal footprint, and thereby control the thermal profile, according to the present invention. The optimization logic is initiated, step 522, including activating the surgical energy device. If quenching is not detected, step 524, then at least one system control setting is increased, step 526, and the surgical site is again observed for quenching, step 524. Once quenching has been detected, the thermal footprint is analyzed, step 528. If the thermal footprint is acceptable, step 530, including at the desired location as discussed above, then a surgical procedure can be performed, step 532. If the thermal footprint is not acceptable, then the same system control setting can be increased again, step 526, or a new system control setting can be adjusted as indicated in phantom for step 534. The logic returns to step 524 in some procedures and, in others, returns directly to step 528 where the altered footprint is analyzed again.

In one sequence of flow chart 520, the sharpness of the thermal profile is increased. First the laser is turned and the system adjusted until quenching is detectable. These adjustments may include any of increasing the power, decreasing the time between pulses or increasing the pulse width, or decreasing the annular fluid flow or increasing the temperature of the annular fluid flow. The sharpness of the profile is analyzed either by the human eye, or via software which measures the slope of the fluorescence versus intensity curve and width of the region of variable fluorescence. If a sharper thermal profile is desired, then the laser system settings may be adjusted until the profile is acceptable for the procedure.

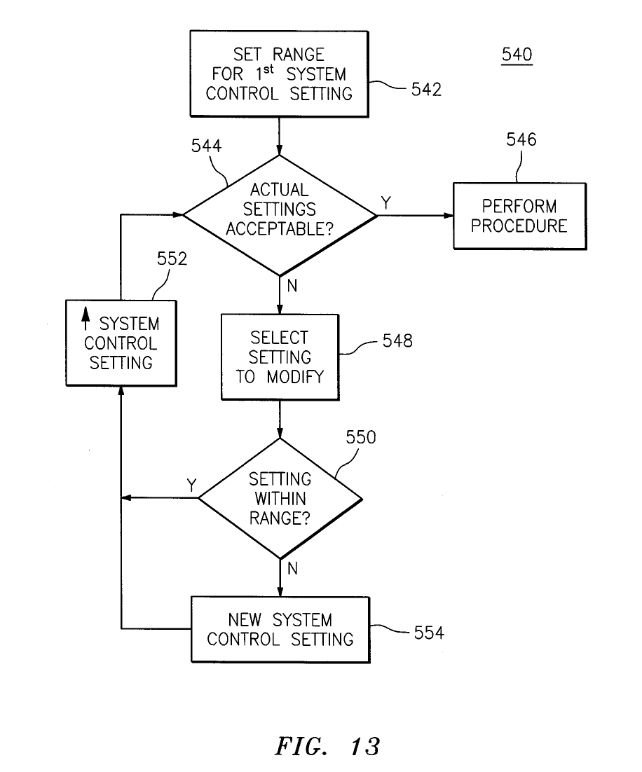

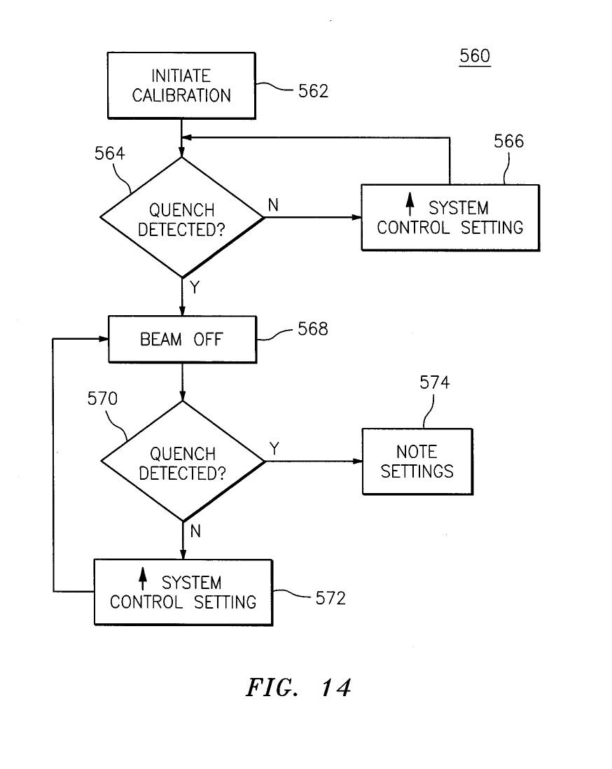

Flow chart 540, FIG. 13, illustrates control of the ranges of values for at least one system control setting. For example, it may be desirable in certain procedures or with certain surgical sites to pre-select certain laser system parameters, or ranges of those parameters, before varying other parameters. For example, the power setting may be fixed, or not to be increased beyond a certain maximum level, and other system control parameters are to be varied thereafter. This control procedure is initiated, step 542, and settings of interest are analyzed, step 544, for actual level or amount, such as temperature or distance ranges. In some constructions, non-optimized footprints can direct the logic to profile optimization flow chart 520, FIG. 12, or to calibration flow chart 560, FIG. 14. If all actual settings are currently acceptable, the logic proceeds to performing the surgical procedure, step 546.

If at least one of the current settings are not acceptable, then at least one setting is selected to be modified, step 548. If the selected setting is within preselected or currently desired ranges of maximum and minimum values for the desired surgical procedure, as determined at step 550, then the selected setting is modified, step 552, and the logic returns to step 544. Preferably, the determination at step 550 that the selected setting is “within” a range allows for at least one incremental increase or decrease in that parameter so the logic can proceed through steps 552 and 554 and still be within the desired range. Otherwise, the logic proceeds to step 544 where another system control setting is selected to be altered, step 552. The logic preferably continues until all current settings are acceptable, step 544 and then surgery is performed, step 546.

Flow chart 560, FIG. 14, illustrates steps to calibrate a surgical energy system according to the present invention. In one implementation, these steps can determine laser system settings for producing irreversible fluorescence changes. The calibration algorithm is initiated, step 562, the laser or other energy device is turned on, and the target location is checked for quenching of fluorescence, step 564. If quenching is not detected, then the energy device is turned off and at least one system control setting is increased, step 566. The energy device is again activated and possible quenching is again observed, step 564. After quenching is detected, the laser is turned off, step 568, and the same target location is again observed, step 570. The settings are adjusted, step 572, and the logic proceeds through steps 568, 570 and 572 until there is a permanent alteration in fluorescence and quenching is no longer detected at step 570. The current settings are noted, recorded or otherwise utilized, step 574, for present or future surgical procedures utilizing that surgical system.

Other surgical procedures utilizing quenching of fluorescence are within the scope of the present invention. It can be useful to the surgeon to create a registration mark in the surgical field inside the patient, as is done sometimes outside the patient before starting a procedure, or as it done sometimes following a biopsy, where a marker is left in place to facilitate navigating to places of interest. In addition, irreversible quenching is thought to indicate irreversible changes in tissue which may occur prior to ablation. It may be desirable to identify laser settings which cause such changes.

Although specific features of the present invention are shown in some drawings and not in others, this is for convenience only, as each feature may be combined with any or all of the other features in accordance with the invention. While there have been shown, described, and pointed out fundamental novel features of the invention as applied to one or more preferred embodiments thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature.

It is the intention, therefore, to be limited only as indicated by the scope of the claims appended hereto. Other embodiments will occur to those skilled in the art and are within the following claims.

What is claimed is:

1. A surgical system, comprising:
a radiation source of at least a first wavelength of illuminating radiation directable to a target location on tissue of a patient at a surgical site to cause at least one type of fluorophore associated with the tissue to fluoresce;
an energy source of at least a first surgical energy directable to the target location and having a plurality of power settings, at least one of the power settings being capable of quenching fluorescence of the fluorophore; and
a processor capable of adjusting at least one of a plurality of system control settings to alter the amount of quenching of the fluorophore, the system control settings including the plurality of power settings.

2. The system of claim 1 wherein the first surgical energy is a beam of electromagnetic radiation.

3. The system of claim 1 wherein the first surgical energy includes a laser beam generated by the energy source.

4. The system of claim 1 wherein the processor includes at least one detector to detect quenching of fluorescence at the surgical site.

5. The system of claim 1 wherein the processor includes at least one image processor capable of generating an image of the surgical site to depict changes in fluorescence.

6. The system of claim 1 wherein the processor includes a controller to adjust the system control settings.

7. The system of claim 6 wherein the controller is capable of moving the surgical energy to a different location on tissue at the surgical site.

8. The system of claim 1 wherein the radiation source provides at least a second wavelength of illuminating radiation to enable a user to see the surgical site.

9. The system of claim 1 further including at least one fluid source having at least two fluid control settings and the system control settings include the fluid control settings.

10. A surgical system, comprising:
a radiation source of at least a first wavelength of illuminating radiation directable to a target location on tissue of a patient at a surgical site to cause at least one type of fluorophore associated with the tissue to fluoresce;
an energy source of at least a first surgical energy beam of electromagnetic radiation directable to the target location and having a plurality of power settings, at least one of the power settings being capable of quenching fluorescence of the fluorophore; and
a processor capable of adjusting at least one of a plurality of system control settings to alter the amount of quenching of the fluorophore, the system control settings including the plurality of power settings, the processor including at least one detector to detect quenching of fluorescence at the surgical site.

11. The system of claim 10 wherein the processor includes at least one image processor capable of generating an image of the surgical site to depict changes in fluorescence.

12. The system of claim 11 wherein the first surgical energy includes a laser beam generated by the energy source.

13. The system of claim 12 wherein the processor includes a controller to adjust the system control settings.

14. The system of claim 13 wherein the radiation source provides at least a second wavelength of illuminating radiation to enable a user to see the surgical site.

15. The system of claim 10 further including at least one fluid source having at least two fluid control settings and the system control settings include the fluid control settings.

16. A method for optimizing treatment of tissue of a patient at a surgical site, comprising:
directing at least a first wavelength of illuminating radiation to a target location on the tissue of the patient to cause at least one type of fluorophore associated with the tissue to fluoresce;
directing at least a first surgical energy from an energy source to the target location at one of a plurality of power settings, at least one of the power settings capable of quenching fluorescence of the fluorophore; and
adjusting at least one of a plurality of system control settings to alter the amount of quenching of the fluorophore, the system control settings including the plurality of power settings.

17. The method of claim 16 wherein adjusting includes observing the location of the quenching and moving the first surgical energy to a different location on tissue at the surgical site.

18. The method of claim 16 further including detecting a change in fluorescence after the at least one system control setting is altered.

19. The method of claim 18 further including modifying treatment of tissue based on the detected change in fluorescence.

20. The method of claim 18 further including estimating the effect on tissue of increasing the at least one system control setting based on the detected change in fluorescence.

21. The method of claim 16 further including directing at least a second wavelength of illuminating radiation, different than the first wavelength, to the surgical site to assist visualization at least substantially at the target location.

22. The method of claim 16 further including utilizing at least one fluid source having at least two fluid control settings, and adjusting includes altering at least one of the fluid control settings as one of the system control settings.
